(19) United States
(12) Patent Application Publication
(10) Pub. No.: US 2020/0178012 A1
(43) Pub. Date: Jun. 4, 2020

(54) NOISE SIMULATING BOOTH FOR SAFELY TESTING AND DEMONSTRATING HEARING PROTECTION EQUIPMENT

(71) Applicant: 3M INNOVATIVE PROPERTIES COMPANY, St. Paul, MN (US)

(72) Inventors: Douglas D. Fletcher, Woodbury, MN (US); Jason T. Lunn, Mahtomedi, MN (US); Gregory G. Jager, Oakdale, MN (US); Oscar M. Hemberg, Dalaro (SE); Eric O. Hemberg, Shatin, NT (HK); Henrik Fransson, Bro (SE)

(21) Appl. No.: 16/088,773

(22) PCT Filed: Mar. 29, 2017
(86) PCT No.: PCT/US2017/024747

§ 371 Date: Sep. 26, 2018

Related U.S. Application Data
(60) Provisional application No. 62/315,723, filed on Mar. 31, 2016.

(51) Int. Cl. H04R 29/00; G06F 3/16; H04R 1/02; G10K 15/04; A61F 11/08; A61F 11/14

(52) U.S. Cl. CPC H04R 29/001; G06F 3/165; A61F 11/14; G10K 15/04; A61F 11/08; H04R 1/025

(57) ABSTRACT

A sound testing chamber comprises a sound proof enclosure, a speaker system and an electronic noise generator system. The speaker system is disposed within the sound proof enclosure. The electronic noise generator system is electronically coupled to the speaker system. The electronic noise generator system is configured to send a signal to the speaker system to generate a sound simulating a scenario where hearing protection is desirable. A method for testing hearing protection equipment using a noise simulating booth comprises: allowing a test subject to select a sound to be played by an electronic noise simulating system; positioning the test subject within a sound proof enclosure having speakers that generate the sound; positioning hearing protection equipment on the test subject; and playing the sound in the sound proof enclosure to permit the test subject to evaluate the hearing protection equipment.

### TECHNICAL FIELD

The present disclosure relates generally, but not by way of limitation, to acoustic testing and evaluating equipment. More specifically, without limitation, this document relates to generating simulated noises is a safe environment.

### BACKGROUND

Equipment for hearing protection typically comprises ear plugs that are inserted in situ into the ear canal to obstruct sound waves, or ear muffs that include sound attenuating material that are placed over the outer ear to block sound waves. The Occupational Safety & Health Administration (OSHA) recommends that hearing protection be used in environments where noise exceeds 85 Decibels (dB) and, thus, is desirable for use during many activities, such as while operating various machines, vehicles, lawn maintenance equipment or firearms.

Ear muffs are additionally advantageous because they provide a platform for including other features, such as audio players (e.g., radios, MP3 players, etc.), sound canceling systems, and active volume control systems.

Hearing protection can be rated on several different factors, such as noise reduction level and “fit” with the customer. Noise reduction in hearing protection is typically governed by ANSI S3.19-1974 in the United States, although various other foreign standards can be used. However, these noise reduction standards may not always be applied appropriately and can vary in hearing protection from different manufacturers. Additionally, compliance with such standards is not always adhered to in hearing protection equipment, particularly in those of dubious origin.

The “fit” of various hearing protection devices, however, cannot be easily reduced to a numerical rating. Thus, consumers are often left to make purchasing decisions for hearing protection based on unsubstantiated packaging claims. The purchasing decision is further complicated by the varying price range for different types of hearing protection, with ear muffs incorporating the aforementioned audio players, sound canceling systems, and active volume control systems being expensive relative to simpler hearing protection solutions such as disposable ear plugs.

Systems for testing hearing equipment are described in U.S. Pat. No. 5,317,273 to Hanson et al., U.S. Pat. No. 5,970,795 to Seidmann et al., and U.S. Pat. No. 7,574,917 to Von Dach et al.

### OVERVIEW

The present inventors have recognized, among other things, various problems associated with prior art systems for testing hearing protection equipment. For example, previous systems for testing hearing equipment comprise hand-held portable devices that can be taken out into the field, which therefore can require that 1) the consumer already own the hearing protection device and 2) the consumer go out and find a noisy environment in which to test the hearing equipment. Also, some systems only allow one particular type of hearing protection device to be tested, i.e., ear plugs or ear muffs. Another shortcoming of some prior art systems is that only the one specific test device can be evaluated, e.g., the testing system cannot be used with any off-the-shelf hearing protection device.

The present disclosure provides a solution to these deficiencies by providing a sound testing and evaluating chamber in which any type of off-the-shelf hearing protection device can be tested. Furthermore, the sound testing chamber can realistically reproduce particular real-world scenarios to allow the customer or user to test and/or evaluate hearing protection devices in a preferred noise situation. Furthermore, a customer or user can evaluate the way various sounds, such as voices, are affected by other louder sounds. One example of this is the ability of the customer or user to evaluate how well his or her hearing recovers after a very loud noise, such as a sonic impulse. Additionally, the sound testing chamber can be a portable, self-contained unit that can be transported to different locations where consumers can test hearing protection devices before purchasing them.

In one embodiment, a sound testing and/or evaluating system comprises: a sound proof enclosure or booth; a speaker system disposed within the sound proof enclosure; and an electronic noise generator system electronically coupled to the speaker system; wherein the electronic noise generator system is configured to send a signal to the speaker system to generate a sound simulating a scenario where hearing protection is desirable.

In another embodiment, a method for testing and/or evaluating hearing protection equipment using a noise simulating demonstration booth comprises: allowing a test subject to select a sound to be played by an electronic noise simulating system; positioning the test subject within a sound proof enclosure having speakers that generate the sound; positioning hearing protection equipment on the test subject; and playing the sound in the sound proof enclosure to permit the test subject to evaluate the hearing protection equipment.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

FIG. 1 is a perspective view of a noise simulating system of the present disclosure comprising a sound proof booth having a speaker system connected to noise generating equipment.

FIG. 2 is a perspective view of the interior of the sound proof booth of FIG. 1 showing the speaker system and an assortment of hearing protection devices.

FIG. 3 is a perspective view of an example wraparound speaker array used in the speaker system of FIG. 2.

FIG. 4A is a schematic front view of the sound proof booth of FIG. 1 showing the height locations of various speaker arrays.

FIG. 4B is a schematic top view of the sound proof booth of FIG. 1 showing the location of the speaker arrays relative to the floor space in the booth.

FIG. 4C is a schematic side view of the sound proof booth of FIG. 1 showing a connector panel.

FIG. 5 is a schematic view of the interior of the sound proof booth of FIG. 4A-4C showing various user interface and safety components.

FIG. 6 is a schematic system view of the sound proof booth, speaker system and noise generating equipment of FIG. 1.

FIG. 7 is a flowchart showing steps in a method for operating the noise simulating system of FIGS. 1-6 in order to ensure the safety of users and operators of the system.

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

### DETAILED DESCRIPTION

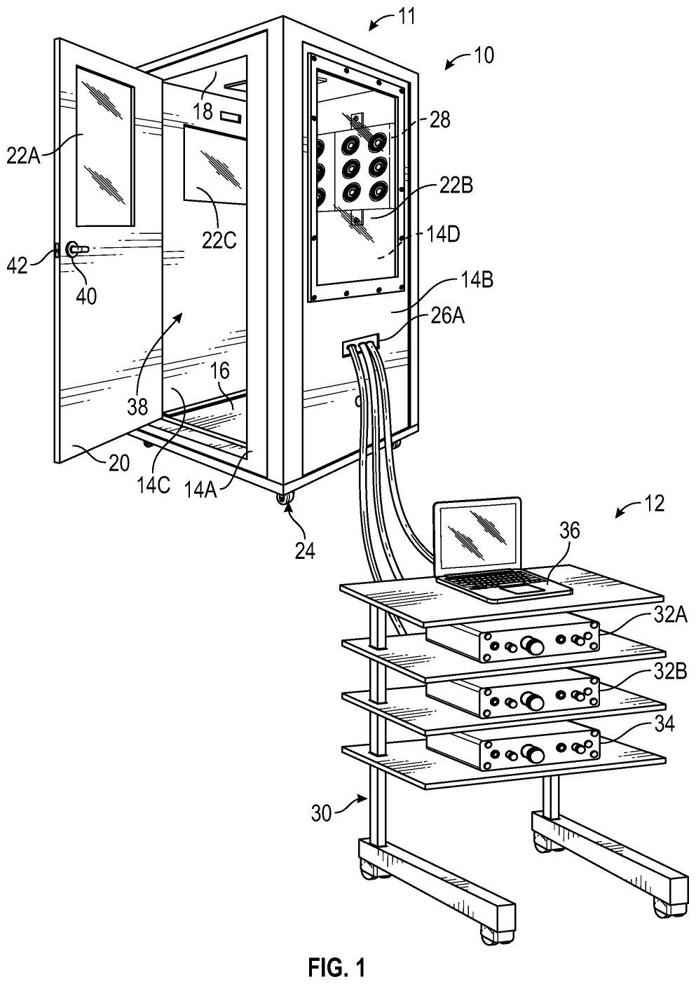

FIG. 1 is a perspective view of a noise simulating system 10 of the present disclosure comprising sound proof booth 11 and sound generator 12. Sound booth 11 includes sidewalls 14A-14D, floor 16, ceiling 18, door 20, windows 22A, 22B and 22C, casters 24, connector panel 26A and speaker system 28. Sound generator 12 includes cart 30, amplifiers 32A and 32B, audio interface 34 and computer 36.

Sound generator 12 is an electronic system configured to produce or generate loud sounds or noises inside sound proof booth 11 that allows an occupant of enclosure 38 to test and/or evaluate hearing protection equipment, while booth 11 prevents the sound from fully escaping into the surrounding environment to protect the hearing of bystanders, such as those operating sound generator 12. Sound generator 12 can produce signals that allow speaker system 28 to produce a variety of single channel and dual channel sounds in order to allow the occupant a simulated experience of actually being in an environment where the loud sounds occur, such as a concert, shooting range or manufacturing environment. Thus, the occupant can evaluate multiple sets of hearing protection equipment protecting against the same sound to determine, based on their own subjective criteria, which hearing protection equipment performs the best for his or her needs. In the described embodiment, sound generator 12 is mounted outside of sound booth 11. However, sound generator 12 can be located in sound booth 11 so as to be accessible either from the outside or inside of sound booth 11. Locating sound generator 12 away from sound booth 11 provides an additional safety feature for allowing operator(s) of sound generator 12 to be located further away from the noise source.

Sound proof booth 11 comprises a sound proof, or nearly sound proof, enclosure 38 that is designed to acoustically isolate an occupant from the environment. Thus, sound originating outside of booth is inhibited from entering enclosure 38, while sound generated within enclosure 38 is inhibited from escaping booth 11. As such, walls 14A-14D, floor 16 and ceiling 18 can include sound absorbing or attenuating material.

Walls 14A-14D and door 20 can include windows that also provide a sound barrier. For examples, windows 22A and 22B can comprise double-paned windows where each panel is separately mounted to booth 11 to reduce vibration, wherein each panel can additionally be laminated further reduce vibration. Booth 11 also includes a plurality of windows such that the occupant of enclosure 38 and the operator of sound generator 12 to have high visibility of each other, thereby reducing the possibility of the operator of sound generator 12 accidentally or undesirably generating noise within booth 11 with the occupant not being prepared, i.e., not wearing hearing protection equipment.

Door 20 can include handle 40 and latch 42. Handle 40 can be operated from within enclosure 38 by an occupant to close door 20 and seal it against the doorframe in wall 14A by engaging latch 42. Handle 40 can include a manually-operated lock on the inside of booth 11 that prevents latch 42 from disengaging the doorframe. As will be discussed in greater detail below, latch 42 can include a sensor connected to computer 36. The sensor, such as a contact sensor, can disable sound generator 12 from producing noise at speaker system 28 unless door 20 is properly closed and sealed. Such a feature protects the safety of both the occupant, by preventing sound from being produced until the occupant is ready, and bystanders, by preventing loud noises generated within enclosure 38 from being allowed to easily escape.

Booth 11 can be configured to be portable. For example, booth 11 can include casters 24 connected to floor 16 that permit booth 11 to easily be transported, such as by rolling. Likewise, cart 30 can include casters (not shown) for easy transporting and moving of sound generator 12. Additionally, sound generator 12 can be sized and configured to be stored within enclosure 38 inside booth 11. For example, individual components of sound generator 12 can be removed from cart 30 and stored on floor 16, with cart 30 being foldable or collapsible to fit within enclosure 38. In other embodiments, sound generator 12 can be integrated into a single unit that can fit through the doorway for door 20 and be disposed within enclosure 38 on floor 16. Cables and wires for sound generator 12 can be detached from connector panel 26A so that the cables and wires can be stored within enclosure 38 along with the rest of sound generator 12.

Computer 36 of sound generator 12 is loaded with various audio files that reproduce environmental noise, such as gunshots and lawn mowing equipment. Computer 36 also includes data that correlates the real-world (i.e., the world as it exists outside the booth) volume for each audio file. For example, it has been determined that a high powered rifle can generate a real-world impulse sound of over 140 dB sound pressure level (SPL). Sound generator 12 includes calibration equipment, as discussed in greater detail below, which permits amplifiers 32A and 32B and audio interface 34 to reproduce the sound in enclosure 38 at the real-world level. As such, a user of noise simulating system 10 can test a plurality of off-the-shelf hearing protection devices in a convenient location (e.g., a retail outlet or trade show) and in a short amount of time in order to conduct their own evaluation of the effectiveness of the devices in an environment where the user intends to utilize the devices.

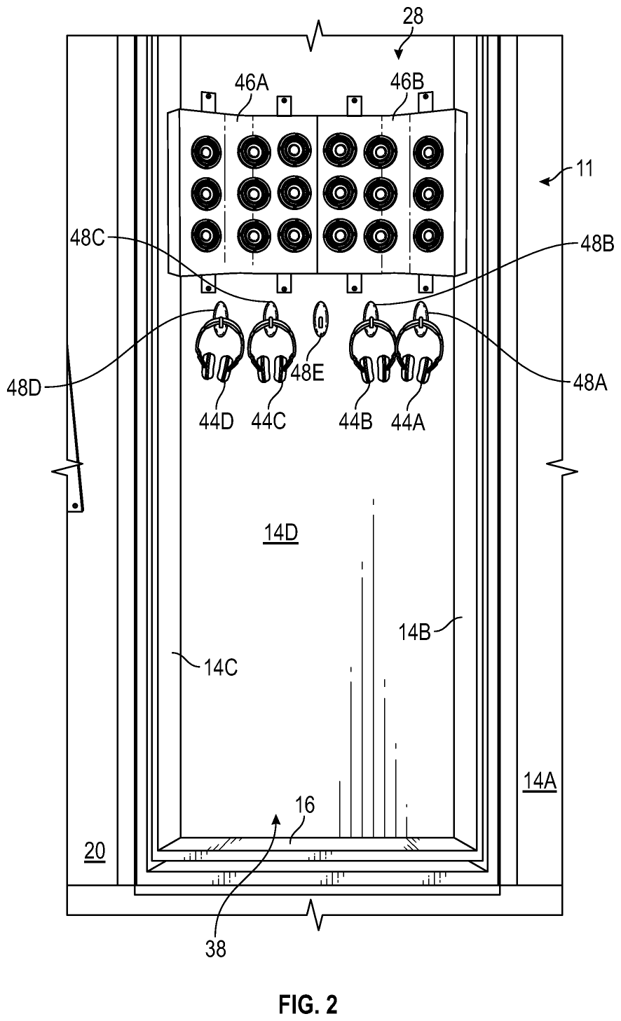

FIG. 2 is a broken-away perspective view of enclosure 38 of sound proof booth 11 of FIG. 1 showing speaker system 28 and an assortment of hearing protection devices 44A-44D. Speaker system 28 includes speaker arrays 46A and 46B.

Hearing protection devices 44A-44D can be suspended from hooks 48A-48E on wall 14D. In the depicted embodiment, hearing protection equipment comprises ear muffs. However, any type of hearing protection equipment, including ear plugs, can be tested and/or evaluated. Hearing protection devices 44A-44D provides the occupant of booth 11 a selection of different hearing protection devices from which to choose in order to evaluate their effectiveness against noise generated by speaker arrays 46A and 46B and sound generator 12. In various examples, hearing protection devices 44A-44D can comprise equipment from different manufacturers, different models of equipment from the same manufacturer, or combinations thereof.

Hearing protection devices 44A-44D can comprise off-the-shelf, unmodified devices. That is, it is not necessary to install any special devices, such as microphones, antennas or wires, on devices 44A-44D, or necessary to make any modifications to hearing protection devices 44A-44D in order to take advantage of the testing and evaluating capabilities of noise simulating system 10. Accordingly, any type of hearing protection equipment can be tested and evaluated with noise simulating system 10, including production prototypes or other hearing protection device evaluation devices.

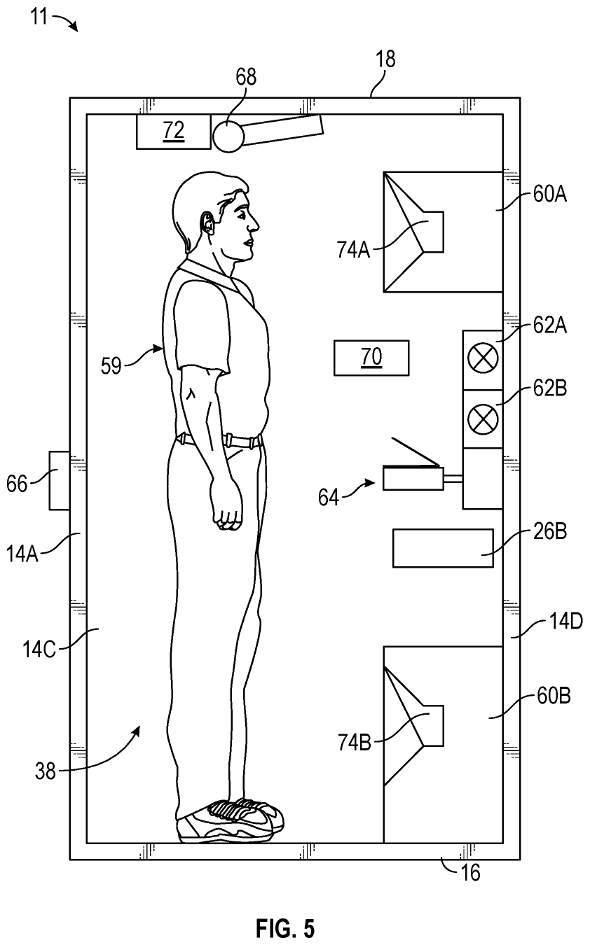

Speaker arrays 46A and 46B are positioned and arranged within enclosure 38 to simulate noise as experienced in the real world for the occupant of booth 11. For example, speaker arrays 46A and 46B are positioned at head level (as shown in FIG. 5). Also, speaker arrays 46A and 46B are shaped so as to, combined, extend across wall 14D and partially extend across walls 14C and 14B. In particular, speaker array 42A extends halfway across wall 14D and partially across wall 14C, while speaker array 42B extends halfway across wall 14D and partially across wall 14B. As such, speaker arrays 42A and 42B provide an optimized and more immersive acoustic experience than a single speaker or flat array might provide for the occupant.

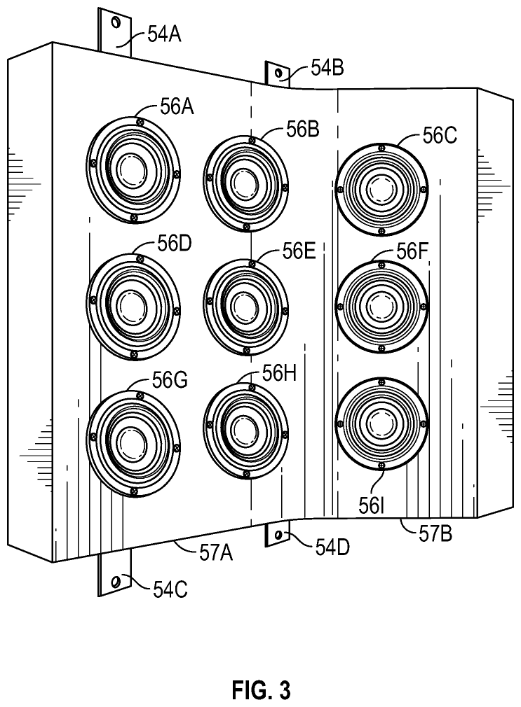

FIG. 3 is a perspective view of wraparound speaker array 50 used in speaker system 28 of FIG. 2. Speaker array 50 includes box 52, brackets 54A-54D and loudspeakers 56A-561.

Box 52 includes base portion 57A and wing portion 57B. Base portion 57A can be mounted along wall 14D, while wing portion 57B can be mounted along wall 14B for the orientation shown, or wall 14C for an orientation rotated one-hundred-eighty degrees from the orientation shown in FIG. 3. Brackets 54A-54D can comprise metal straps that can be used to secure speaker array 50 to walls 14A-14D of booth 11.

Box 52 provides a cabinet for loudspeakers 56A-561 that allow loudspeakers 56A-561 to move air and generate bass. A large number of loudspeakers 56A-561 (e.g., eighteen in FIG. 2) is provided to generate a large amount of bass so that an occupant of enclosure 38 can feel the sound being generated. Also, a large number of loudspeakers 56A-561 is provided to facilitate rapid responsiveness and high frequency sound production.

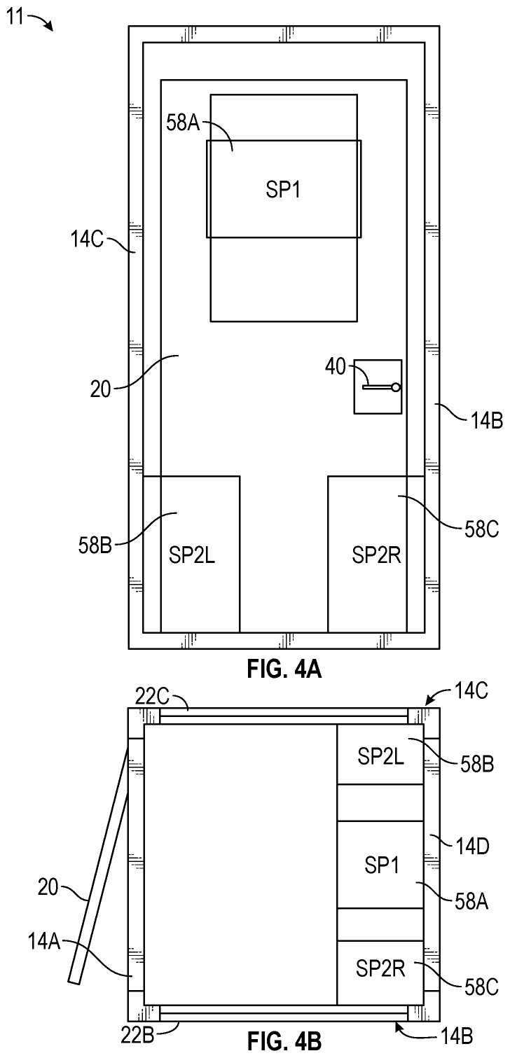

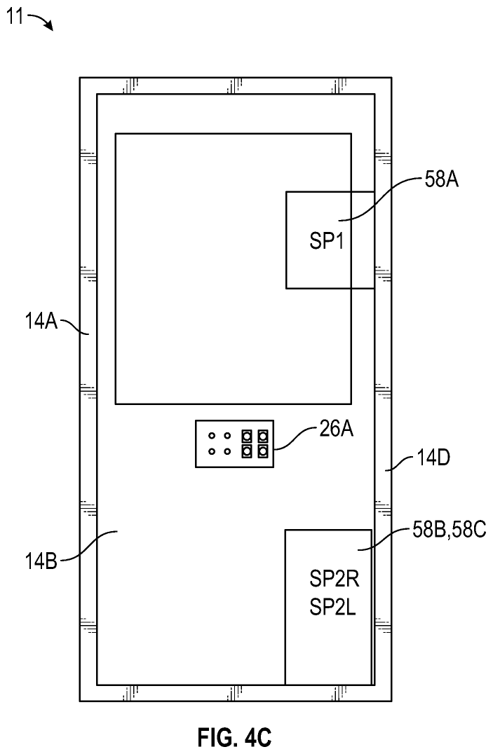

Speaker array 50 can be used as speaker arrays 46A and 46B of FIG. 2, or speaker arrays 58A-58C of FIG. 4A or speaker arrays 60A and 60B of FIG. 5.

FIG. 4A is a schematic front view of another embodiment of noise simulating system 10 of FIG. 1 showing the height locations of various speaker arrays 58A-58C within booth 11. FIG. 4B is a schematic top view of noise simulating system 10 of FIG. 1 showing the location of speaker arrays 58A-58C relative to floor 16 of booth 11. FIG. 4C is a schematic side view of booth 11 of FIG. 1 showing connector panel 26A.

Sound proof booth 11 includes similar components as shown and discussed with reference to FIGS. 1-3. However, booth 11 of FIGS. 4A-4C includes speaker arrays 58A-58C rather than speaker arrays 46A and 46B of FIG. 2. Speaker arrays 58A-58C are arranged differently than speaker arrays 46A and 46B to provide a different surround sound experience.

Speaker array 58A is positioned at head level within enclosure 38 and is centered on wall 14D. Speaker arrays 58B and 58C are positioned at a lower level than array 58A. This allows speaker arrays 58A-58C to simulate directional noises when testing hearing protection equipment. For example, speaker arrays 58B and 58C can simulate equipment noise, such as that from a riding lawnmower deck positioned below head level, while speaker array 58A can simulate a human voice, such as that from another person talking to the person on the riding lawnmower.

In another example, speaker arrays 58B and 58C can be positioned along wall 14A opposite speaker array 58A. For example, speaker array 58B can be positioned at the corner of wall 14A and wall 14C, and speaker array 58C can be positioned at the corner of wall 14A and wall 14B at the level of the head of the occupant. Such an arrangement of speaker arrays 58A-58C can simulate a firing line or shooting range scenario where loud gunshot noises are simulated in front of the occupant, while voice commands and instructions are simulated behind the occupant. In one example, speaker arrays 58A-58C can be removably mounted within enclosure 38 so that they can be mounted in different locations to better simulate different scenarios. Speaker arrays 58A-58B can thus be mounted with releasable fasteners, such as threaded fasteners, hooks or hook and loop material.

Speaker arrays of the present system, however, need not be positioned at different levels to produce two separate channels of audio, as speaker array 46A can be used to produce a separate sound from speaker array 46B if desired. In other examples, two or more audio files playing a plurality of different sounds can be played simultaneously using all speaker arrays.

FIG. 5 is a schematic view of the interior of sound proof booth 11 of FIG. 4A-4C showing user 59 relative to various user interface components. In FIG. 5, 11 includes connector panel 26B, upper speaker array 60A, lower speaker array 60B, first lamp 62A, second lamp 62B, activation button 64, door switch 66, microphone 68, user interface 70 and calibration mechanism 72.

Upper speaker array 60A and lower speaker array 60B can include one or more loudspeakers, such as loudspeakers 74A and 74B, respectively, and can function similarly as to other speaker arrays described herein. For example, loudspeakers 74A and 74B can be spatially positioned within enclosure 38 to provide a surround sound experience from a plurality of audio channels.

Activation button 64 can be connected to computer 36 through connector panel 26B via any suitable wired or wireless connection. Activation button 64 comprises a user-activated button that allows user 59 to lock-out or prevent sound generator 12 from causing speaker arrays 60A and 60B to generate sound or noise within enclosure 38. In one embodiment, activation button 64 prevents computer 36 from sending an electronic audio signal to speaker arrays 60A and 60B. In another embodiment, activation button 64 can prevent an operator of sound generator 12 from playing an audio file with computer 36. In yet another embodiment, activation button 64 can prevent amplifiers 32A and 32B from generating power. In still another embodiment, activation button 64 can be connected to speaker arrays 60A and 60B to prevent speaker arrays 60A and 60B from receiving a signal from sound generator 12. Activation button 64 can be a “hold-closed” switch meaning that user 59 must continuously engage activation button 64 to disable sound generator 12; when activation button is released, sound generator 12 is enabled. Alternatively, activation button 64 can be a “hold-open” switch meaning that user 59 must continuously engage activation button 64 to enable sound generator 12; when activation button is released, speaker arrays 60A and 60B are prevented from playing sound. From a safety standpoint, a “hold-open” switch is advantageous because sound can only be played when user 59 is consciously ready for it.

Door switch 66 can be connected to computer 36 through connector panel 26B via any suitable wired or wireless connection. Door switch 66 can comprise a door-activated switch that locks-out or prevents sound generator 12 from causing speaker arrays 60A and 60B from generating sound or noise within enclosure 38. Door switch 66 can disable speaker arrays 60A and 60B and/or sound generator 12 in a similar manner as activation button 64. Door switch 66 is activated when door 20 is not fully closed, such as when door 20 is open and latch 42 is not fully engaged, thereby preventing sound generated by speaker arrays 60A and 60B from escaping enclosure 38 and potentially causing harm or discomfort to bystanders. Door switch 66 is inactive when door 20 is fully closed against the doorframe in wall 14A such that latch 42 is fully seated, thereby ensuring that speaker arrays 60A and 60B will only generate sound when it can be contained by booth 11. In one embodiment, door switch 66 comprises a magnetic contact switch with one or more components mounted on door 20 and/or the doorframe.

First lamp 62A and second lamp 62B comprise visual indicators that allow user 59 and the operator of sound generator 12 to know the status of noise simulating system 10. For example, lamps 62A and 62B can emit different colored light to indicate if system 10 is armed or not. In particular, first lamp 62A can emit a red light if either activation switch 64 is not actuated (user 59 not ready) or door switch 66 is activated (door 20 is open), while second lamp 62B can emit a green light if both activation switch 64 is actuated (user 59 ready) or door switch 66 is inactive (door 20 is closed). Other colors or flashing patterns can be used to provide an indication of other events, states, activities or statuses. For example, a yellow light or a flashing light can be used to indicated that noise is about to be generated by speaker arrays 60A and 60B. In one embodiment, lamps 62A and 62B comprise light emitting diodes (LEDs) connected to computer 36 through connector panel 26B via any suitable wired or wireless connection.

User interface 70 comprises a device that allows user 59 to interact with computer 36 in order to control various aspects of the noise generated by speaker arrays 60A and 60B. For example, user interface 70 can comprise a laptop computer or a tablet computer that allows user 59 to see menus and lists of information and to select items from those menus and lists. In one embodiment, user interface 70 allows user 59 to select from a list of noises to be generated by sound generator 12. In various embodiments, user interface 70 provides a text display of the name of the noise to be played or that is currently playing. In another embodiment, user interface 70 displays the volume level (e.g., dB level) of the noise to be played or that is currently playing speaker arrays 60A and 60B.

Microphone 68 and calibration mechanism, here calibration tool 72, together allow speaker arrays 60A and 60B to provide noises within enclosure 38 at realistic or real-world levels. For example, calibration tool 72 is configured to produce a tone that corresponds to a test dB level that is to be generated by speaker arrays 60A and 60B. When calibration tool 72 plays the tone, microphone 68 sends a signal to computer 36 that corresponds to the test dB level, indicating what magnitude of signal correlates to the test dB level. Calibration tool 72 is a portable, hand-held sized device that can be removed from booth 11 after the calibration process is completed. Calibration tool 72 can be configured to mechanically couple to microphone 68 in a known or predetermined way such that the tone generated by calibration tool 72 will be received or heard in the exact same way every time by microphone 68. With calibration tool 72 removed, computer 36 can cause sound to be generated by speaker arrays 60A and 60B until microphone 68 again registers the test dB level and sends the corresponding signal to computer 36. Thus, computer 36 will know what level of output is needed from amplifiers 32A and 32B to cause the test dB level. Subsequently, audio files of computer 36 can be played at predetermined dB levels stored in computer 36, as discussed below, using the test dB level as a baseline.

Computer 36 can also be provided with audio files that are to be played at predetermined dB levels in enclosure 38. For example, computer 36 can have stored therein an audio file of a lawnmower operating or of a firearm discharging, as well as a particular level of noise that is correlated with each specific sound. The correlated recorded sounds (audio files) for each specific sound can be stored in computer 36 in a database where the corresponding particular dB level is matched to each audio file. Thus, when the audio file for the specific sound is played, computer 36 also knows what dB level should be detected by microphone 68 in enclosure 38 and can adjust output from amplifiers 32A and 32B according to the calibration process.

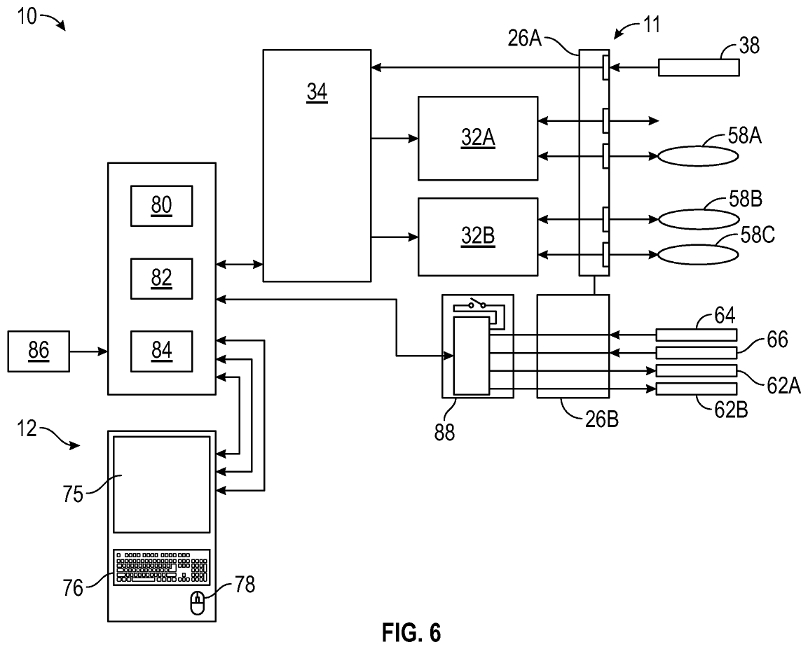

FIG. 6 is a schematic system view of noise simulating system 10 of FIG. 1 including sound proof booth 11 and sound generator 12. Sound generator 12 includes computer 36, which includes display screen 75, keyboard 76, mouse 78, processor 80, working memory 82, stored data 84 and removable storage device 86.

Computer 36 can be any computer capable of properly operating an audio player capable of playing digitally recorded audio files and related software, such as a computer similar to a commercially available personal computer that comprises processor 80, working memory 82, stored data 84 and removable storage device 86. Processor 80 can be a processor of sufficient power for computer 36 to perform desired functions, such as one or more microprocessors. Working memory 82 can be memory sufficient for computer 36 to perform desired functions such as solid-state memory, random-access memory, and the like. In one embodiment, computer 36 comprises a portable laptop computer.

Stored data 84 can be any program configured to operate sound generator 12, including the aforementioned audio files, which can include MP3, WAV, WMA, AAC, RIFF, or any other suitable audio file format. Stored data 84 can also include other software for editing audio files, such as for adjusting output levels, adjusting bass and treble, applying effects and filters, adjusting stereo channels and the like. Stored data 84 can also include audio files previously recorded, such as those for producing noise sounds within booth 11.

Computer 36 can include a variety of input devices, such as keyboard 76 and mouse 78. Any other conventional computer peripheral, such as pointing devices, cameras, microphones, printers and scanners, can be used. Computer 36 can include a variety of output devices, such as display screen 75, other visual output devices, audio output devices such as speakers and the like.

Removable storage device 86 can be any device having a removable storage media that would allow downloading data, such as audio files of various noises and sounds. The removable storage device can be a read-write compact disc (CD) drive, a read-write digital video disc (DVD) drive, a flash solid-state memory port, a removable hard drive, a floppy disc drive, and the like. Removable storage device 86 can include audio files having recordings of noises to be played in booth 11.

Audio interface 34 comprises a hardware component that enables computer 36 to play audio files at desirable levels and qualities and can include additional input-output ports for connecting to microphones, musical instruments and other devices. Audio interface 34 allows for adjustment in the playback of audio files in order to achieve high quality resolution and fidelity, as well as to adjust the authenticity of noise generated in booth 11. In one example, audio interface 34 receives played audio file signal from computer 36, conditions the signal, and then sends it to amplifiers 32A and 32B for transmission to speaker arrays 58A-58C.

Amplifiers 32A and 32B comprise conventional audio amplifiers that are commercially available and that are capable of increasing the power of audio signals generated by computer 36 and audio interface 34. In one embodiment, each amplifier 32A and 32B is rated at 2×1500 Watts into 2 Ohms or 2×900 Watts into 4 Ohms. In one example, amplifiers 32A and 32B are capable of producing sounds within enclosure 38 at levels of up to 140 dB of sound pressure level (SPL) for impulse sounds and up to 130 dB SPL for constant sounds, which is set in order to ensure the safety of user 59 and bystanders. As such, system 10 can be configured to play higher (louder) sound levels in enclosure 38 in other applications, such as for non-human testing of hearing protection equipment with microphones. Two amplifiers are provided with sound generator 12 in order to receive two separate signals from computer 36 that are ultimately transmitted to speaker arrays 58A-58C. Thus, two different noises or sounds from different audio channels can be played in booth 11 on different speaker arrays.

As described previously, components of sound proof booth 11, such as microphone 38, speaker arrays 58A-58C, lamps 62A and 62B, activation button 64 and door switch 66 can be connected to components of sound generator 12 at panels 26A and 26B. Safety devices, such as lamps 62A and 62B, activation button 64 and door switch 66, can also be connected to computer 36 via a master safety switch 88. Furthermore, power can be supplied to all components of system 10, such as computer 36, audio interface 34 and amplifiers 32A and 32B, display screen 75 and lamps 62A and 62B, via a master power system and switch, which is not illustrated for simplicity.

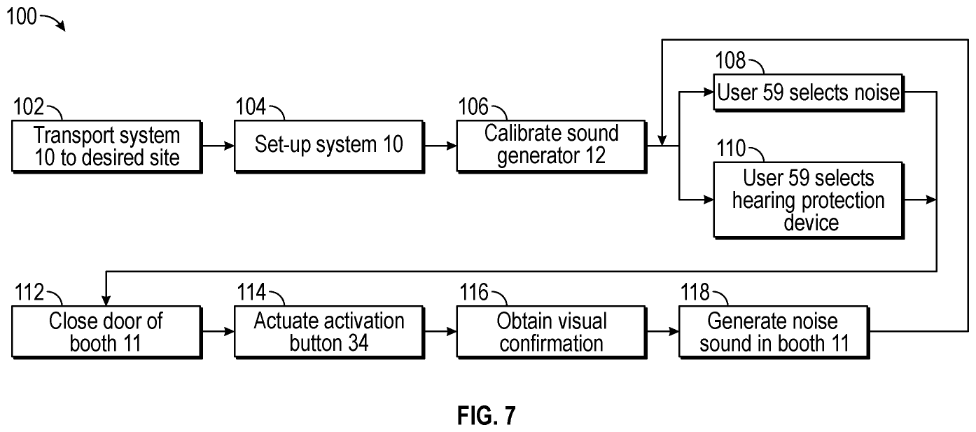

FIG. 7 is a flowchart showing steps in method 100 for operating noise simulating system 10. Steps 102-118 are shown in an order that helps ensure the safety of the occupant of booth 11 as well as bystanders, such as the operator of sound generator 12. In other embodiments, however, the steps can be performed in different orders so long as all safety procedures are followed.

At step 102, sound proof booth 11, with sound generator 12 disposed within enclosure 38 can be transported to a desired location, such as a retail store, a trade show, a manufacturing facility, or any other desired location.

At step 104, sound generator 12 can be removed from inside sound booth 10. Any wires from sound generator 12 can be connected to connector panel 26A from the outside of booth 11 or connector panel 26B on the inside of booth 11. Sound generator 12 is positioned outside of booth 11 so that an operator of sound generator 12 can view an occupant of booth 10 through one of windows 22A-22C while operating computer 36. Sound booth 10 can be positioned using casters 24 and sound generator 12 can be positioned using casters on cart 30.

Next, at step 106, sound generator 12 can be calibrated using microphone 68 and calibration tool 72 as previously described.

At step 108, user 59 can then select from a menu of noises available on computer 36 to be played by speaker arrays 58A-58C. Computer 36 can be provided with audio files of any sound, including common noises and voices talking. Common noises can be stored in working memory 82, stored data 84 and removable storage device 86, and can include noise from a plurality of different sources, such as gun shots, lawnmowers, construction equipment, crowd noise such as from a sporting event, dogs barking and the like.

At step 110, user 59 then enters enclosure 38 of booth 11 and selects one of hearing protection devices 44A-44D to be worn for evaluation. Any type of hearing protection equipment can be worn and evaluated, including ear plugs and ear muffs.

After user 59 equips the selected hearing protection equipment, door 20 is closed at step 112 using handle 40 (FIG. 1) such that latch 42 fully engages the doorframe and door switch 66 is inactivated.

Next, at step 114, user 59 can grasp activation button 64 to activate it and enable sound generator 12 to produce noise within chamber 38.

At step 116, an operator of sound generator 12 can then obtain visual confirmation from user 59 through windows 22A-22C that user 59 has properly seated the selected hearing protection device and is ready for noise to be produced by speaker arrays 58A-58C.

Operator of sound generator 12 can then operate computer 36 to generate the noise within booth 11 at step 118. After the noise is played and user 59 is satisfied with the evaluation of the selected hearing protection device, user 59 can release activation button 64, disabling speaker arrays 58A-58C from producing noise, and select a different hearing protection device to be evaluated. User 59 can then equip a different one of hearing protection devices 44A-44D and then provide a visual indication to the operator of sound generator 12 through windows 22A-22C that user 59 is again ready for noise to be generated.

In one embodiment, user 59 can open door 20 between instances of noise generation to have the operator of sound generator 12 select a different audio file for playback. In another example, user 59 can remain in booth 11 with door 20 closed and can select different audio files for playback using user interface 70. In one embodiment, user interface 70 is not capable of actually causing sound generator to produce noise at speaker arrays 58A-58C to help ensure that noise will be generated only when all safety measures have been verified by the operator of sound generator 12.

### VARIOUS NOTES & EXAMPLES

Example 1 can include or use subject matter (such as a sound testing system comprising: a sound proof enclosure; a speaker system disposed within the sound proof enclosure; and an electronic noise generator system disposed outside of the sound proof enclosure and electronically coupled to the speaker system; wherein the electronic noise generator system is configured to send a signal to the speaker system to generate a sound simulating a scenario where hearing protection is desirable.

Example 2 can include, or can optionally be combined with the subject matter of Example 1, to optionally include a sound proof enclosure and an electronic noise generator that are portable.

Example 3 can include, or can optionally be combined with the subject matter of one or any combination of Examples 1 or 2 to optionally include a sound proof enclosure that is mounted on casters and an electronic noise generator that can be stowed within the sound proof enclosure.

Example 4 can include, or can optionally be combined with the subject matter of one or any combination of Examples 1 through 3 to optionally include a door in the sound proof enclosure that is lockable from the inside; and a switch that disables the electronic noise generator system unless the door is latched.

Example 5 can include, or can optionally be combined with the subject matter of one or any combination of Examples 1 through 4 to optionally include an electronic noise generator system that is prevented from sending the signal by the switch unless the door is locked.

Example 6 can include, or can optionally be combined with the subject matter of one or any combination of Examples 1 through 5 to optionally include an activation button located in the sound proof enclosure that must be actuated before the electronic noise generator system is enabled to send the signal.

Example 7 can include, or can optionally be combined with the subject matter of one or any combination of Examples 1 through 6 to optionally include an activation button that must be continuously actuated by a user in order for the electronic noise generator system to send the signal.

Example 8 can include, or can optionally be combined with the subject matter of one or any combination of Examples 1 through 7 to optionally include an electronic noise generator system that further comprises: an amplifier for generating the sound; an audio player for generating the signal; and a volume control for controlling a magnitude of the sound.

Example 9 can include, or can optionally be combined with the subject matter of one or any combination of Examples 1 through 8 to optionally include an electronic noise generator system that further comprises: a display for showing a list of sounds capable of being played by the audio player; and a user interface for selecting a sound from the list.

Example 10 can include, or can optionally be combined with the subject matter of one or any combination of Examples 1 through 9 to optionally include an electronic noise generator system that is mounted on a cart.

Example 11 can include, or can optionally be combined with the subject matter of one or any combination of Examples 1 through 10 to optionally include a user controller disposed within the sound proof chamber and electronically coupled to the electronic noise generator system, the user controller configured to select the sound generated by the electronic noise generator system.

Example 12 can include, or can optionally be combined with the subject matter of one or any combination of Examples 1 through 11 to optionally include a user controller that can be locked-out by the electronic noise generator system.

Example 13 can include, or can optionally be combined with the subject matter of one or any combination of Examples 1 through 12 to optionally include a user controller that further comprises a display for showing a name of the sound selected by the user controller and an audio level of the sound selected by the user controller.

Example 14 can include, or can optionally be combined with the subject matter of one or any combination of Examples 1 through 13 to optionally include indicator lights within the sound proof enclosure that indicate if the door is latched and/or if the activation button is actuated.

Example 15 can include, or can optionally be combined with the subject matter of one or any combination of Examples 1 through 14 to optionally include a microphone; and a calibration mechanism; wherein the electronic noise generator system and speaker system are configured to simulate sounds in the isolation chamber at audio levels commensurate with those of the sounds in the real world.

Example 16 can include, or can optionally be combined with the subject matter of one or any combination of Examples 1 through 15 to optionally include a speaker system that includes speakers on first and second channels, and wherein the electronic noise generator system is configured to send first and second signals to the first and second channels, respectively, to generate two separate sounds.

Example 17 can include, or can optionally be combined with the subject matter of one or any combination of Examples 1 through 16 to optionally include a speaker system that includes a first speaker array that is mounted at head level within the sound proof chamber and that is configured to at least partially wrap around the sound proof enclosure, the first speaker array connected to receive the first signal.

Example 18 can include, or can optionally be combined with the subject matter of one or any combination of Examples 1 through 17 to optionally include a speaker system that includes a second speaker array mounted at a different spatial location within the sound proof chamber than the first speaker array, the second speaker array connected to receive the second signal.

Example 19 can include, or can optionally be combined with the subject matter of one or any combination of Examples 1 through 18 to optionally include an electronic noise generator system that generates sounds selected from the group comprising gunshots, construction noise, lawnmower noise and voices talking.

Example 20 can include, or can optionally be combined with the subject matter of one or any combination of Examples 1 through 19 to optionally include an electronic noise generator system and speaker system that are capable of generating constant sound up to approximately 130 dB SPL.

Example 21 can include, or can optionally be combined with the subject matter of one or any combination of Examples 1 through 20 to optionally include an electronic noise generator system and speaker system are capable of generating impulse sounds up to approximately 140 dB SPL.

Example 22 can include, or can optionally be combined with the subject matter of one or any combination of Examples 1 through 21 to optionally include an assortment of hearing protection equipment located within the sound proof enclosure.

Example 23 can include or use subject matter such as a method for testing hearing protection equipment using a noise simulating demonstration booth, the method comprising: allowing a test subject to select a sound to be played by an electronic noise simulating system; positioning the test subject within a sound proof enclosure having speakers that generate the sound; positioning hearing protection equipment on the test subject; and playing the sound in the sound proof enclosure to permit the test subject to evaluate the hearing protection equipment.

Example 24 can include, or can optionally be combined with the subject matter of Example 23, to optionally include verifying the safety of the test subject and the environment of the sound proof enclosure.

Example 25 can include, or can optionally be combined with the subject matter of one or any combination of Examples 23 or 24 to optionally include verifying the safety of the environment of the sound proof enclosure to include verifying that a door of the sound proof enclosure is latched before playing the sound.

Example 26 can include, or can optionally be combined with the subject matter of one or any combination of Examples 23 through 25 to optionally include verifying the safety of the test subject to include activating a button within the sound proof enclosure by the test subject before playing the sound.

Example 27 can include, or can optionally be combined with the subject matter of one or any combination of Examples 23 through 26 to optionally include calibrating a volume level of the sound to produce a simulated sound in the sound proof enclosure at an audio level commensurate with that of the simulated sound in the real world.

Example 28 can include, or can optionally be combined with the subject matter of one or any combination of Examples 23 through 27 to optionally include playing a simulated voice simultaneously with the sound in the sound proof enclosure on a separate channel from the sound.

Example 29 can include, or can optionally be combined with the subject matter of one or any combination of Examples 23 through 28 to optionally include generating the sound at a spatially disparate locations within the sound proof enclosure.

Example 30 can include, or can optionally be combined with the subject matter of one or any combination of Examples 23 through 29 to optionally include the test subject comparing operation of different hearing protection equipment within the sound proof enclosure.

Example 31 can include, or can optionally be combined with the subject matter of one or any combination of Examples 23 through 30 to optionally include allowing the test subject to evaluate a first hearing protection device from a first manufacturer; and allowing the test subject to evaluate a second hearing protection device of a second manufacturer.

Example 32 can include, or can optionally be combined with the subject matter of one or any combination of Examples 23 through 31 to optionally include showing performance differences to the test subject of the first and second hearing protection devices based on the evaluations.

Example 33 can include, or can optionally be combined with the subject matter of one or any combination of Examples 23 through 32 to optionally include transporting the sound proof enclosure to a location where the test subject can be selected from a group of potential purchasers of the hearing protection equipment.

Example 34 can include, or can optionally be combined with the subject matter of one or any combination of Examples 23 through 33 to optionally include directing the test subject to a point of sale for hearing protection equipment after the test subject evaluates the hearing protection equipment.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A sound testing system comprising:
a sound proof enclosure;
a speaker system disposed within the sound proof enclosure; and
an electronic noise generator system electronically coupled to the speaker system;
wherein the electronic noise generator system is configured to send a signal to the speaker system to generate a sound simulating a scenario where hearing protection is desirable.

2. The sound testing system of claim 1, wherein the sound proof enclosure and the electronic noise generator are portable.

3. The sound testing system of claim 1, further comprising:
a door in the sound proof enclosure that is lockable from the inside; and
a switch that disables the electronic noise generator system unless the door is latched.

4. The sound testing system of claim 1, further comprising an activation button located in the sound proof enclosure that must be actuated before the electronic noise generator system is enabled to send the signal.

5. The sound testing system of claim 4, wherein the activation button must be continuously actuated by a user in order for the electronic noise generator system to send the signal.

6. The sound testing system of claim 1, wherein the electronic noise generator system further comprises:
an amplifier for generating the sound;
an audio player for generating the signal; and
a volume control for controlling a magnitude of the sound.

7. The sound testing system of claim 6, wherein the electronic noise generator system further comprises:
a display for showing a list of sounds capable of being played by the audio player; and
a user interface for selecting a sound from the list.

8. The sound testing system of claim 1, further comprising a user controller disposed within the sound proof chamber and electronically coupled to the electronic noise generator system, the user controller configured to select the sound generated by the electronic noise generator system.

9. The sound testing system of claim 1, wherein the user controller can be locked-out by the electronic noise generator system.

10. The sound testing system of claim 1, further comprising:
a microphone; and
a calibration mechanism;
wherein the electronic noise generator system and speaker system are configured to simulate sounds in the isolation chamber at audio levels commensurate with those of the sounds in the real world.

11. The sound testing system of claim 1, wherein the speaker system includes speakers on first and second channels, and wherein the electronic noise generator system is configured to send first and second signals to the first and second channels, respectively, to generate two separate sounds.

12. The sound testing system of claim 11, wherein the speaker system includes a first speaker array that is mounted at head level within the sound proof chamber and that is configured to at least partially wrap around the sound proof enclosure, the first speaker array connected to receive the first signal.

13. The sound testing system of claim 11, wherein the speaker system includes a second speaker array mounted at a different spatial location within the sound proof chamber than the first speaker array, the second speaker array connected to receive the second signal.

14. (canceled)

15. A method for testing hearing protection equipment using a noise simulating demonstration booth, the method comprising:
allowing a test subject to select a sound to be played by an electronic noise simulating system;
positioning the test subject within a sound proof enclosure having speakers that generate the sound;
positioning hearing protection equipment on the test subject; and
playing the sound in the sound proof enclosure to permit the test subject to evaluate the hearing protection equipment.

16. The method of claim 15, further comprising verifying the safety of at least one of the test subject and the environment of the sound proof enclosure.

17. The method of claim 16, wherein verifying the safety of the environment of the sound proof enclosure comprises verifying that a door of the sound proof enclosure is latched before playing the sound, and wherein verifying the safety of the test subject comprises activating a button within the sound proof enclosure by the test subject before playing the sound.

18. The method of claim 15, further comprising calibrating a volume level of the sound to produce a simulated sound in the sound proof enclosure at an audio level commensurate with that of the simulated sound in the real world.

19. The method of claim 15, further comprising playing a simulated voice simultaneously with the sound in the sound proof enclosure on a separate channel from the sound.

20. The method of claim 15, further comprising generating the sound at a spatially disparate locations within the sound proof enclosure.

21. The method of claim 15, further comprising:
allowing the test subject to evaluate a first hearing protection device from a first manufacturer;
allowing the test subject to evaluate a second hearing protection device of a second manufacturer; and showing performance differences to the test subject of the first and second hearing protection devices based on the evaluations.
